(19) United States
(12) Patent Application Publication
(10) Pub. No.: US 2005/0211590 A1
(43) Pub. Date: Sep. 29, 2005

(54) Protective cover for medical devices

(76) Inventors: George Kent McClure, Georgetown (CA); Margaret Ann Johnston, Belmont (CA)

Correspondence Address: TIM HEADLEY, GARDERE WYNNE SEWELL LLP, 1000 LOUISIANA, SUITE 3400, HOUSTON, TX, 77002, US

(21) Appl. No.: 10/811,500

(22) Filed: Mar. 26, 2004

(51) Int. Cl. A61L 15/00; A61B 19/02; A61B 17/06; G01N 1/00; G01N 35/00

(52) U.S. Cl. 206/438; 206/441

(57) ABSTRACT

A removable protective housing is used to cover an exposed portion of a medical device extending from a body. The cover includes a first housing with an opening formed along a single edge of the housing. The edge intersects a longitudinal axis defined by the exposed portion of the device. The edge may be sealed to enclose the exposed portion of the device within the housing. The housing may be made of flexible materials such as impermeable fabrics. A spring may bound the opening to preferentially move the edge into a closed position. The edge may also include a band of adhesive to releasably seal the edge around an entry point through which the device extends out from the body. A kit for covering the device may includes two housings. A first housing includes a sealable edge to enclose the exposed portion within the first housing. The first housing fits within the second housing. The second housing is secured to the body, around the entry point. The second housing may be secured to the body by an adhesive band. The two housings may be made of impermeable fabrics.

### FIELD OF THE INVENTION

The present invention relates to protective covers for medical devices including catheters, gastrointestinal tubes, nephrostomey tubes, abscess drains, common bile duct tubes, feeding tubes, stomach feeds and other partially exposed devices which may be implanted in or during medical procedures.

### BACKGROUND

In the medical arts, many kinds of medical devices are used in connection with health care procedures and treatment programs. By way of example, central catheters, of which there are many examples of varying designs, are partially implanted into a patient so that a portion is lodged within the body, specifically such that the tip is inserted into the bottom two-thirds of the superior venacava (which is the largest vein in the human body). These catheters are often used in a variety of medical treatments, such as by way of example, hemodialysis, stem cell retrieval and some chemotherapies.

After the catheter or other medical device is implanted, a portion of the catheter (or other device) is exposed, outside of the patient's body. The exposed portion of the catheter, or other medical device, should be kept dry, clean and protected to provide comfort and avoid contamination or other accidental injury to the patient. In the past, nurses and other medical staff have devised make-shift dressings and coverings, typically using gauze and medical adhesive tapes to prepare protective wrappings about the exposed devices. Although such makeshift devices may provide some protective benefits, various problems may arise. Makeshift coverings are prone to substantial variation due to differences introduced by individual application techniques, variations found in the medical supplies used to make the coverings and other factors. By way of example, these earlier coverings are difficult to remove with the combination of adhesive tape and gauze dressing adhering to the patient so that upon removal of the coverings, the catheter or other medical device could pull away from the patient causing pain, injury and additional long term discomfort and other possible complications to the patient.

These earlier coverings were made of materials which did not offer protection against water contact or wetting of the medical devices, the incision or the opening in the body through which the catheter or other device was introduced into the patient's body. Although the medical supply materials used to make these makeshift coverings were often sterile, they were typically porous, water absorbent or water permeable materials, and did not offer protection against wetting or contamination.

In one earlier catheter cover made of a porous, water permeable cotton fabric, the cotton fabric was sewn into a two-walled pouch having ‘hook and loop’ type fastening bands (for example VELCRO™ brand fasteners) along two adjacent, outer edges of the cover. This known example is illustrated in FIG. 2. In this illustration, a catheter 1 extends outwardly from a patient's body, through an entry point 11 in the patient's chest 8. A sterile dressing 19 covers the exit site 11 and catheter 1, the sterile dressing securing the exposed portion 3 of the catheter 1 to the patient's chest. Catheter tubes 7 extend outwardly away from the body, toward their distal tips 9. The hook and loop fastening bands 42, 42′ and 47, 47′ could be opened so that the exposed portion 3 of the catheter 1 could be introduced into the interior of the cover 41. The cover 41 was made of a breathable and permeable fabric, namely cotton. The upper fabric layer 43 was sewn to the underlying fabric layer 44 along two adjoining margins. The two opposing edges 45 and 46 of the cover, opposing the two closed margins, were edged with hook and loop fastening features to allow the user to open the cover and insert the exposed portion of the catheter. In this device, the two edges 45 and 46 defined by adjoining hook and loop segments 42, 42′ and 47, 47′ defined a single opening. The cover 41, with the catheter positioned within the interior space of the cover, would then be closed by engaging the opposing bands of the hook and loop fasteners (along 42, 42′ and 47, 47′). Often a medical care provider would be required to use both hands to disengage the hook and loop fastening bands and thereafter remove the protective cover without tugging or dislodging the catheter. Medical staff, and those patients who were confident enough to remove the covers themselves, would often need both hands to firmly grip and then carefully open or close the earlier protective covers including these hook and loop fasteners.

### SUMMARY OF THE INVENTION

In one aspect, the invention includes a removable protective cover for an exposed portion of a medical device extending outwardly from a body. The outwardly extending medical device defines a longitudinal axis. The cover comprises a housing that defines an opening along a single edge of the housing. The edge defines a line that intersects the axis. The edge may be sealed to enclose the exposed portion.

In one embodiment, the housing may include a pair of opposing walls that define a sealable pocket. The opposing walls may be made of one or more flexible materials, for example, one or more fabrics. The flexible materials may be impermeable to water, and contaminants including pathogens.

In a preferred embodiment, the edge may be operated between an open position and a closed position by use of a single hand. For example, a spring may be positioned adjacent the edge to releasably seal the opening.

In another aspect, the single edge lies in a plane. The edge forms a releasable seal when in contact with the body. The plane may include an adhesive suitable for contact with exposed skin on the body. The adhesive may form a band that surrounds an entry point through which the medical device extends into the body. It is preferred that the adhesive secures the cover to the body, to form a barrier about the entry point.

The cover may include a display for treatment information. For example, the display may comprise a transparent pocket for display of a patient's name, special instructions or other information relating to the patient's medical treatment. In another example, the display may be a surface on which ink writing may be applied by medical staff. Other display elements and techniques may be provided.

In another preferred embodiment, the invention is a kit including two housings used to cover an exposed portion of a medical device extending outwardly from a body. The first housing defines an opening along an edge. That edge is sealable so that the exposed portion of that medical device is enclosed within the first housing. The second housing receives the first housing. Preferably, the second housing completely envelopes the first housing. The second housing is releasably secured to the body about an entry point through which the medical device extends from the body. One or both of the housings may be made from flexible materials such as fabrics. The flexible materials are preferably impermeable to water and contaminants. The second housing may include a band for releasably securing the second housing directly to the body. For example, the band may include an adhesive suitable for direct contact with exposed skin on the body. It is preferable that the adhesive band be applied to the body in a manner that will provide an impermeable barrier about the entry point.

The entry point may be a small incision created during a medical procedure. The entry point may be partially protected with a dressing. Often the dressing will include gauze and other air permeable and water absorbent fabrics. Often the dressing will be air permeable, to allow oxygen to contact the wound, to inhibit infection or other detrimental effects. In addition, the dressings will often include absorbent portions to allow absorption of fluids leaking or oozing from the surgically created entry point.

In some embodiments, the first housing may be visible through the second housing. For example, the second housing may be made from a substantially transparent material, for example, a flexible, transparent film of thermoplastic material so that the position and condition of the first housing may be readily determined, without having to remove or disturb the second housing. One of the first or second housings or both may include a display for treatment information.

In a preferred kit, the first housing will include a flexible band about the opening. The flexible band may act as a spring to bias the edge of the housing toward the closed position. The band may also include a deformable seal to tightly grip about the exposed portion of the medical device. The deformable seal may be provided by opposing segments that tightly mate or engage to inhibit inward migration of water and contaminants. One or both opposing segments may be made of deformable foam having a memory so that preferably, the foam will return to its original state when disengaged from the medical device.

The protective cover of the present invention may provide one or more of the following advantages or other advantages which will become apparent upon a review of the present specification. By way of an example, one or more of the following advantages may be obtained:
an easily removable protective cover may be provided to enclose an exposed portion of a medical device secured to a body;
certain embodiments of the protective cover may be removable with the use of a single hand;
certain embodiments of the protective cover may be made of water resistant or water impermeable materials;
certain embodiments of the protective cover may provide a barrier against contamination of the exposed portion of the medical device and/or the opening through which the device is introduced into the body;
a kit may be provided in which a first removable protective cover may be used to enclose an exposed portion of the medical device, and a second removable protective cover may be provided to inhibit contamination of an opening through which a medical device is introduced into the body; and
one or more of these advantages, or other advantages, may be available to those who use or provide embodiments of the present invention.

The foregoing are only some examples of certain embodiments of the invention. Many other embodiments, variations and derivations will become apparent from a review of the entire specification, including the description and appended drawings.

### IN THE DRAWINGS

Certain specific embodiments of the invention will be described with reference to the following drawings in which:

FIG. 1 is a top view, in perspective, of a first embodiment of the invention partially enclosing an exposed catheter implanted in a patient.

FIG. 2 is an enlarged top view of an earlier version of a catheter cover in the prior art, in partially opened position, exposing a partially enclosed catheter.

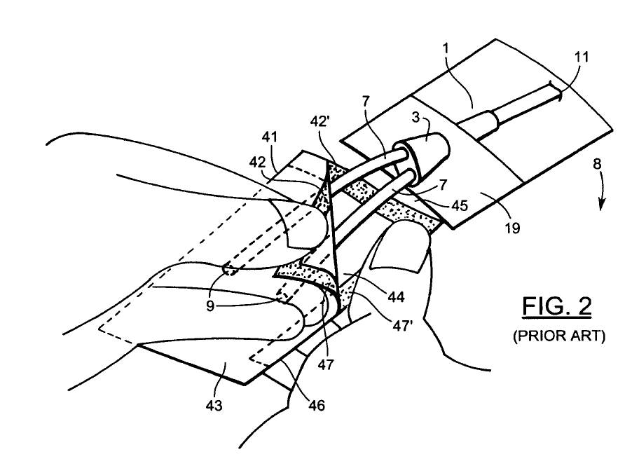

FIG. 3 is an enlarged top view of an embodiment of the present invention showing a self-sealing catheter cover.

### DETAILED DESCRIPTION OF CERTAIN EMBODIMENTS OF THE INVENTION

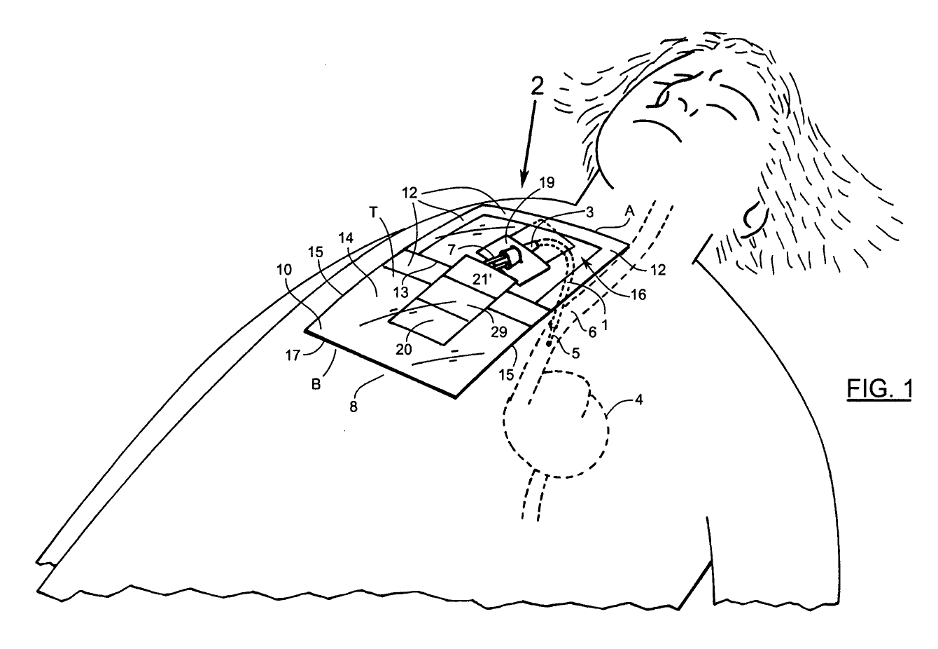

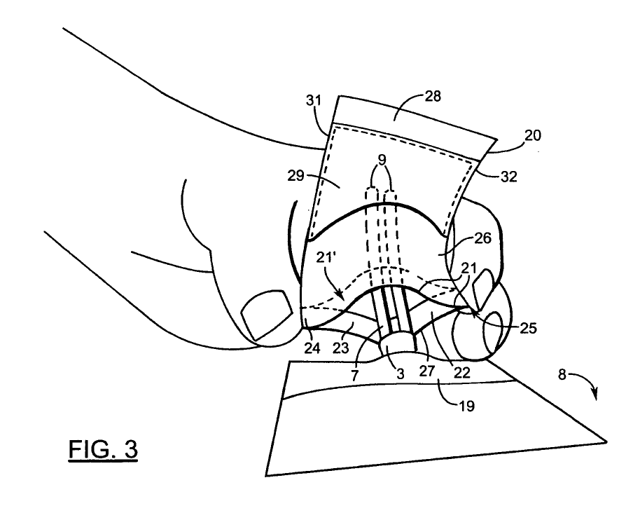

A preferred embodiment of the invention is shown in FIG. 1. Specifically, in this embodiment, a kit is shown for covering a catheter 1 extending into a patient's body 2. An exposed portion 3 of the catheter 1 is covered by a sterile dressing 19 (including adhesive tape) secured to the patient's body. The dressing 19 is applied over an entry point (such as an incision) into the chest wall of the patient's body. In this example, the proximal tip 5 of the catheter 1 extends into the superior venacava 6 through which blood flows via the heart 4. Catheter tubes 7 extend into the interior of an inner housing 20. Inner housing 20 provides a clamping pocket to protect the distal ends 9 of catheter tubes 7 as further illustrated in FIG. 3.

When the catheter 1 is implanted in this manner, the distal ends 9 of the catheter tubes 7 would be exposed, on the exterior of the patient's body, perhaps covered with a dressing gown or a layer of gauze or other dressing material. The inner housing 20 may be applied to cover the distal tips 9 of the catheter 1 together with an additional dressing (not shown), if such an additional dressing is present or desired. In the preferred embodiment as further illustrated in FIG. 3, the housing 20 is opened by applying pressure at opposing grips 24, 25 and squeezing apart upper edge portion 21 away from lower edge portion 23, to form a first opening 22. Preferably, edge 21′ is provided with a clamping feature, such as for example, two opposing spring members secured within upper and lower edge portions 21, 23. The spring members (not shown) may be secured adjacent edge 21′, to bias the housing toward the closed position, so that the opening 22 is closed, and edge portions 21, 23 are securely clamped across tubes 7, thus covering previously distal tips 9 of the catheter 1.

In this preferred embodiment, the inner clamping pocket (or inner housing) 20 is provided with two opposing walls, specifically first wall 26 and second wall 27 secured together along side margins 31, 32 and along opposite margin 28. Information pocket 29 is provided on the exterior of wall 26 to display the patient's medical treatment information. That information may include the patient's name, personal information, information concerning drug dosages, or other medical treatment information. The information pocket 29 may be a transparent compartment that may receive printed information on a card, paper or other device, for easy viewing by attending medical personnel. This display feature may be provided in other ways. For example, the medical treatment information may be supported on a writing surface applied to an outer portion of wall 26. In some embodiments, the display will be reusable, to allow additions or changes to the displayed information, and in other instances, the display may be designed for single usage.

One or both of walls 26, 27 may be made of transparent material. A variety of suitable materials will be apparent to those skilled in the relevant art. In some instances, it may be desirable to use a breathable fabric, such as a pretreated cotton fabric. In many instances, including preferred embodiments, it may be more desirable that the walls 26, 27 be made of an impermeable material to prevent contamination of the distal tips 9 of the catheter tubes 7.

In another embodiment of the invention, the opposing margin 28 may be opened and closed in a manner similar to the steps for manipulating clamping edge 21′. For example, in this modified embodiment, the opposing margin 28 will define a separate opening, fitted with a separate clamping feature similar to the spring members described above with reference to clamping edge 21′ shown in FIG. 3. In a preferred version of this example, the opposing margin 28 may be provided with clamping features which mirror those features of clamping edge 21′, to make the respective edges 21′ and 28 interchangeable, or to allow access to the interior of the housing 20 without requiring removal of the housing 20 from the exposed portion of the catheter 1.

In FIG. 1, the kit includes both an inner housing 20 and an outer, or second, housing 10. In some embodiments of the invention, it may be desirable to forego use of the second housing 10. It may be useful to utilize only housing 20 to provide a protective outer covering over the exposed tips 9 of the catheter 1. In other instances, it may be desirable to forego the use of the inner housing 20, and to apply only the outer housing 10 over the exposed distal tips 9 of the catheter.

However, where a kit with two housings is used, or the outer housing 10 is used without housing 20, the features of housing 10 may be illustrated with reference to FIG. 1. As shown in FIG. 1, an outer housing 10 is attached to the chest 8 of the patient's body 2 using an adhesive band 12 which surrounds an opening 16 into the interior of the housing 10. The interior of the housing 10 defines a pocket 14 to receive the housing 20, which in turn surrounds the exposed tips 9 of the tubes 7. The pocket 14 is bounded by an upper wall T and a lower wall B, the lower wall being shown in an intermediate position between upper wall T and the patient's chest 8. The upper wall T is bounded by lower margin 17, side margins 15, side portions of the adhesive band 12 and upper margin A as shown in FIG. 1. The lower wall B is bounded by lower margin 17, side walls 15 and inner edge portion 13.

When first manufactured, the adhesive band 12 may be covered with a removable protective film or layer that may be peeled off, to expose the adhesive band, for attachment to the patient's body. Of course, other variations will be readily apparent to those skilled in the art after reading this specification.

With reference to the example in FIG. 1, the adhesive band 12 lies in a single plane, to define a single opening 16 to the interior pocket 14. When viewed from a side elevation, the single plane defines a single edge (or for example, a single line) for access to the opening 16. When applied over the exposed portion of the catheter, and when the housing 10 is so viewed from its side, the single plane intersects the catheter across the longitudinal axis of the catheter.

The adhesive band runs along side band portions 12, inner edge portion 13 and along upper margin A. The opening 16 is sealed against the environment, by inserting the exposed portion of the catheter 1 (either with or without the first housing 20) and then applying the adhesive band to the patient's skin so that the adhesive band sticks to the patient's chest.

The housing 10 may be made from various materials that would be suitable for use in medical applications. For example, the walls T, B may be made from the same or different materials. One or both of the walls may be transparent, to allow easy viewing of the interior of the housing, including the contents of pocket 14. Walls T, B may be made of impermeable materials to prevent contamination of the exposed distal tips 9 of catheter tubes 7. It is also preferable that the materials of construction will be selected and manufactured so that the resulting article will be “sterile”. The size and shape of the opening 16 may be designed to allow a user to place the opening 16 of housing 10 in a position above an entry point into the patient's body (similar to another entry point as shown in FIG. 1.)

In some instances, it may be desirable that the housing 10 be made of a fabric which prevents entry of water droplets (for example when the patient washes or showers) but will be sufficiently permeable to allow air (and most importantly oxygen) to reach the entry point, to inhibit infections or other harm to the underlying tissue. The choice of fabric or other materials of construction will depend on a variety of factors which are understood by those skilled in the art.

Housing 10 may also be provided with a display for medical treatment information, similar to the display features described above with reference to housing 20, including with reference to FIG. 3.

Housing 10 may also be provided with a second distinct opening at lower margin 17. For example, in some embodiments, it may be desirable to allow access to the interior of the housing 10 without requiring the user to disengage or remove the housing from the patient's chest. In this modified embodiment, it may be desirable to provide a reusable opening and closing feature. For example, the lower margin 17 may be provided with a closing clamp similar to the clamping feature described with reference to clamping edge 21′ of housing 20. Of course, other suitable closures may be provided if desired. For example, where the housing is made of two thermoplastic film layers, the supplementary closure at lower margin 17 may take the form of interlocking zipper-like track members (of the kind shown for example in Canadian Patent 1,062,207 issued Sept. 11, 1979 entitled Reclosable Plastic Bag Construction Made From A One Piece Extrusion).

Although the foregoing examples have been described in terms of two sided pockets, other shapes and configurations of protective housings and covers are possible. In addition, although the examples were described in terms of medical treatments of humans, certain embodiments of the invention will also be useful with medical devices used in certain veterinary applications.

The invention also includes a method of providing a resealable protective cover for medical devices. By way of example, the invention includes a method of providing a removable, resealable sterile protective cover for an exposed portion of a medical device extending along a longitudinal axis from a body, the cover comprising a housing, the housing defining an opening along a single edge, the method comprising:
introducing the exposed portion across the single edge, into the housing;
positioning the exposed portion of the medical device within the housing so that the edge
intersects the axis;
sealing the edge to enclose the exposed portion within the housing; and
securing the housing relative to the body.

In the method of the invention, the housing may be secured relative to the body via an adhesive band. The adhesive band may be provided as a layer or coating of adhesive material that is pre-applied along the single edge. In other embodiments, the housing may be secured relative to the body by releasing a biasing member positioned along the single edge to engage the exposed portion of the medical device.

The foregoing are examples of certain aspects of the present invention. Many other embodiments, including modifications and variations thereof, are also possible and will become apparent to those skilled in the art upon a review of the invention as described herein. Accordingly, all suitable modifications, variations and equivalents may be resorted to, and such modifications, variations and equivalents are intended to fall within the scope of the invention as described herein and within the scope of any issued patent claims.

1. A removable protective cover for an exposed portion of a medical device extending along a longitudinal axis from a body, the cover comprising:
a housing defining an opening along a single edge to receive the exposed portion, the edge intersecting the axis, and the edge being sealable to enclose the exposed portion within the housing.

2. The cover claimed in claim 1, the housing comprising a pair of opposing walls defining a sealable pocket.

3. The cover claimed in claim 2, the pocket being made of one or more flexible materials.

4. The cover claimed in claim 3, the one or more flexible materials being water impermeable.

5. The cover claimed in claim 1, the edge being operable between a closed position and an open position by operation of a single hand.

6. The cover claimed in claim 1, the edge being biased for operation between a closed position and an open position.

7. The cover claimed in claim 6, the housing comprising a spring for biasing the edge for movement from the open position to the closed position.

8. The cover claimed in claim 1 comprising adhesive along the first edge to releasably seal the opening.

9. The cover claimed in claim 1, the edge being defined along a single plane in sealable contact with the body.

10. The cover claimed in claim 9, the plane comprising a band of adhesive encircling the opening for releasably securing the cover directly to the body, about an entry point through which the medical device extends into the body.

11. The cover claimed in claim 9, the plane comprising a band for releasably securing the cover directly to the body.

12. The cover claimed in claim 11, the band comprising an adhesive to secure the cover to the body, thereby forming a barrier about the entry point.

13. The cover claimed in claim 1 further comprising a display for treatment information.

14. A kit for covering an exposed portion of a medical device extending outwardly from a body, the kit comprising:
a first housing defining an opening along an edge, the edge being sealable to enclose the exposed portion within the first housing; and
a second housing receiving the first housing, the second housing being releasably securable to the body, about an entry point through which the medical device extends into the body.

15. The kit claimed in claim 14, the first housing defining a pocket comprising a first set of two opposing walls.

16. The kit claimed in claim 15, the first set of opposing walls being made of one or more flexible materials.

17. The kit claimed in claim 16, the one or more flexible materials forming an impermeable barrier.

18. The kit claimed in claim 14, the second housing comprising a band for releasably securing the second housing directly to the body.

19. The kit claimed in claim 18, the band comprising an adhesive to secure the second housing directly to the body, about an entry point though which the medical device extends from the body.

20. The kit claimed in claim 19, the band forming an impermeable barrier about the entry point.

21. The kit claimed in claim 20, the second housing comprising a second set of opposing walls made from one or more flexible materials to form an impermeable barrier.

22. The kit claimed in claim 14, the first housing being visible through the second housing when the two housings are secured to the body.

23. The cover claimed in claim 1, the housing defining a second opening opposite the single edge, the second opening being reclosable when the protective cover is secured to the body.

24. The cover claimed in claim 23, the second opening being defined along a second single edge, the second edge intersecting the axis, and the second edge being sealable to enclose the exposed portion within the housing.

25. A method of providing a removable, resealable sterile protective cover for an exposed portion of a medical device extending along a longitudinal axis from a body, the cover comprising a housing, the housing defining an opening along a single edge, the method comprising:
introducing the exposed portion across the single edge, into the housing;
positioning the exposed portion of the medical device within the housing so that the edge intersects the axis;
sealing the edge to enclose the exposed portion within the housing; and
securing the housing relative to the body.

26. In the method claimed in claim 25, comprising the step of securing the housing to the body via an adhesive band about the opening.

27. In the method claimed in claim 25, comprising the step of securing the housing relative to the body by releasing a biasing member positioned along the single edge to engage the exposed portion.
